(19) United States
(12) Patent Application Publication
(10) Pub. No.: US 2018/0143128 A1
(43) Pub. Date: May 24, 2018

(54) RESIDUAL TOXICANT DETECTION DEVICE

(71) Applicant: INDUSTRIAL TECHNOLOGY RESEARCH INSTITUTE, Hsinchu (TW)

(72) Inventors: Jui-Hung Tsai, Hsinchu (TW); Ying-Hao Wang, Hsinchu (TW); Chia-Jung Chang, Hsinchu (TW); Chih-Hao Hsu, Hsinchu (TW); Jing-Yuan Lin, Hsinchu (TW)

(21) Appl. No.: 15/813,264

(22) Filed: Nov. 15, 2017

Related U.S. Application Data
(60) Provisional application No. 62/423,818, filed on Nov. 18, 2016.
(60) Provisional application No. 62/516,192, filed on Jun. 7, 2017.

(30) Foreign Application Priority Data
Feb. 15, 2017 (TW) 106104870
Sep. 30, 2017 (TW) 106133914

(51) Int. Cl. G01N 21/31; G01N 21/01; G01N 21/94; G01N 33/02; G01N 33/18

(52) U.S. Cl. CPC G01N 21/31; G01N 21/01; G01N 21/94; G01N 2021/7783; G01N 33/1826; G01N 2021/3125; G01N 33/025

(57) ABSTRACT

Disclosed is a residual toxicant detection device for detecting the amount of residual toxicants in an aqueous solution to be measured. The residual toxicant detection device includes an accommodation space formed from a shell, a water inlet and a water outlet positioned on the shell for the aqueous solution to flow therein and thereout, respectively, a sensing chamber in the accommodation space, a light source emitter and a light sensor positioned near the sensing chamber, the light source emitter emitting light of a wavelength range, the light sensor receiving the light passing through the sensing chamber, and a circuit board receiving sensing signals sensed by the light sensor, such that absorbance and a change of the absorbance of residual toxicants in the aqueous solution to be measured are calculated.

### CROSS-REFERENCE TO RELATED APPLICATION

The present disclosure is based on, and claims priority from Taiwan Application No. 106104870, filed Feb. 15, 2017, Taiwan Application No. 106133914, filed Sep. 30, 2017, U.S. Provisional Application No. 62/423,818, filed Nov. 18, 2016, and U.S. Provisional Application No. 62/516,192, filed Jun. 7, 2017, the disclosure of which are hereby incorporated by reference herein in its entirety.

### BACKGROUND

### 1. Technical Field

The present disclosure relates to a residual toxicant detection device that detects the change of residual toxicants in an aqueous solution.

### 2. Description of Related Art

Existing food testing is generally based on inspection methods. In the current environment where food safety is of high importance, the detection of residual toxicants has become very important. For example, fruits and vegetables may have pesticide residues, but few people test their fruits and vegetables for pesticide residues.

At present, common methods of detection of pesticides generally include biochemical and spectroscopic methods. The biochemical detection involves biochemical enzyme inhibition method, in which a fixed sample is required for biochemical reaction. This type of method is difficult for consumers to carry out at home. Moreover, the existing biochemical method can only detect pesticides, such as organic phosphorus and carbamate pesticides, without the capability of detecting all pesticides currently used. Besides, this method may produce potentially inaccurate results (false negative −35%, pseudo-positive −50%). This method may not be effective in detecting pesticides, especially for illegal pesticides. In view of the needs for home testing by the general public, the biochemical method requires a considerable amount of complex procedures comparable to laboratory procedures. This renders the method impractical for general home-testing applications.

In the spectroscopic method, spectral comparison is performed to determine the types of pesticides and their concentrations. However, there are currently more than 300 kinds of pesticides registered in the database, and, as such, a large number of comparisons need to be made, which led to higher detection difficulty. In addition, the use of this method inevitably calls for quantitative sampling, which is difficult for the general public to carry out. Moreover, LC/MS/MS (i.e. liquid chromatography (LC) and mass spectrometer (MS) used in series) or high performance liquid chromatography (HPLC) approaches are not widely used, since they are expensive and time consuming.

Therefore, there is a need for a detection technique that is suitable for home testing and effectively monitors the removal of residual toxicants without damaging the test subject and without requiring quantitative sampling.

### SUMMARY

The present disclosure provides a residual toxicant detection device for detecting a residual toxicant in an aqueous solution to be measured. The residual toxicant detection device may include: a shell including an accommodation space; a water inlet formed on the shell and in communication with the accommodation space to allow the aqueous solution to be measured to flow into the accommodation space; a sensing chamber provided in the accommodation space, allowing the aqueous solution to be measured to flow therein; a light source emitter provided near the sensing chamber for emitting light passing through the sensing chamber; a light sensor provided near the sensing chamber for sensing the light passing through the sensing chamber to produce a sensing signal; a water outlet formed on the shell and in communication with the accommodation space to allow the aqueous solution to be measured to flow out of the accommodation space; and a circuit board electrically connected with the light sensor for receiving the sensing signal.

### BRIEF DESCRIPTION OF THE DRAWINGS

FIGS. 1A and 1B are schematic diagrams depicting the overall appearance and an exploded view of a residual toxicant detection device in accordance with the present disclosure;

FIG. 2 is a schematic diagram depicting the internal flow channel of the residual toxicant detection device in accordance with the present disclosure;

FIGS. 3A and 3B are schematic diagrams depicting the outer casing of the residual toxicant detection device in accordance with the present disclosure; and

FIGS. 4A and 4B are schematic diagrams depicting the overall appearance and an exploded view of a mount for the residual toxicant detection device in accordance with the present disclosure.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

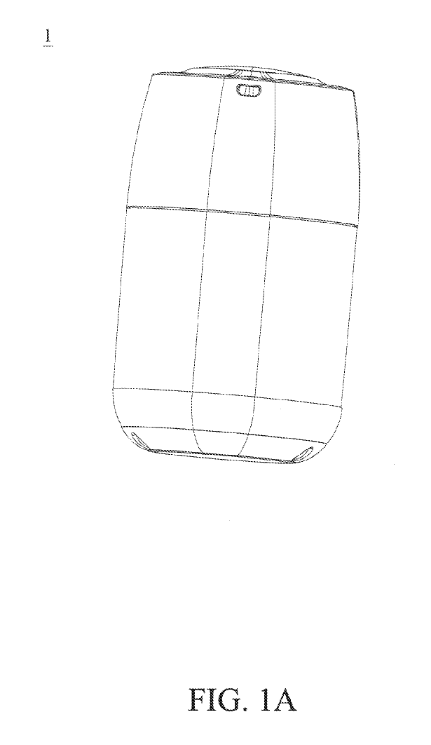

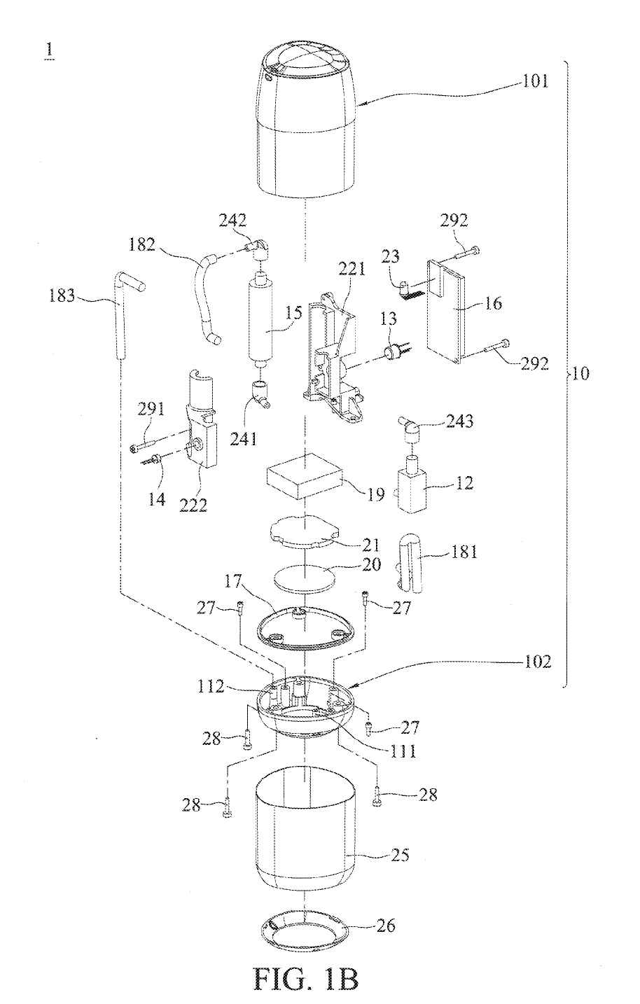

In an embodiment, referring to FIGS. 1A and 1B, schematic diagrams depicting the overall appearance and an exploded view of a residual toxicant detection device in accordance with the present disclosure are shown. As shown in FIG. 1, the residual toxicant detection device 1 in accordance with the present disclosure has a hand-held size and appears to be cylindrical. Two holes are provided near the bottom of the cylinder for an aqueous solution to be measured to flow in and out, respectively. The residual toxicant detection device 1 can be placed in an aqueous solution to be measured to detect absorbance of residual toxicants in the aqueous solution to be measured.

As shown in FIG. 1B, the exploded view of the residual toxicant detection device 1 shows a shell 10, a water inlet 111, a water outlet 112, a sensing chamber 12, a light source emitter 13, a light sensor 14, a pump 15 and a circuit board 16.

The shell 10 is a hollow shell constituting the outside of the residual toxicant detection device 1 for providing waterproof functionality for the residual toxicant detection device 1. An accommodation space is formed in the shell 10 for receiving the sensing chamber 12, the light source emitter 13, the light sensor 14, the pump 15 and the circuit board 16 therein. The shell 10 is made of a waterproof material.

In an implementation, the shell 10 includes an upper lid 101 and a lower lid 102. The upper and the lower lids 101 and 102 join together to form the accommodation space.

The water inlet 111 is positioned on the shell 10 to allow the aqueous solution to be measured to flow into the accommodation space. The water outlet 112 is also positioned on the shell 10 to allow the aqueous solution to be measured to flow out of the accommodation space. If the combination of the upper and the lower lids 101 and 102 is used, the water inlet 111 and the water outlet 112 can be provided on the lower lid 102. Moreover, when the upper and the lower lids 101 and 102 are joined, the residual toxicant detection device 1 is sealed, except for where the water inlet 111 and the water outlet 112 are that allow the aqueous solution to be measured to flow in and out.

In addition, the water inlet 111 and the water outlet 112 are preferably positioned at two opposite far ends of the lower lid 102 to avoid liquid coming out of the water outlet 112 to be sucked back into the water inlet 111, affecting the continuous detection of the absorbance of the residual toxicants in the aqueous solution to be measured.

The sensing chamber 12 is positioned in the accommodation space of the shell 10 to allow the aqueous solution to be measured to flow therein. Simply put, the aqueous solution to be measured, after entering the residual toxicant detection device 1 through the water inlet 111, will pass through the sensing chamber 12, in which the absorbance of the residual toxicants in the aqueous solution to be measured will be tested. The sensing chamber 12 can be made of quartz or fused silica, for example.

The light source emitter 13 and the light sensor 14 can be provided near the sensing chamber 12. The light source emitter 13 is used for emitting light of a specific wavelength range. The light sensor 14 is used for sensing the light of the wavelength range passing through the sensing chamber 12. As mentioned before, the present disclosure provides an optical sensing mechanism, in which the light source emitter 13 and the light sensor 14 are provided near the sensing chamber 12, such that the absorbance of the residual toxicants in the aqueous solution to be measured can be determined based on the emission and sensing of light of a specific wavelength range.

In order to detect multiple toxicants, the light source emitter 13 can be an ultraviolet (UV) LED. In addition, the light emitted by the light source emitter 13 may have different specific wavelength ranges. In an embodiment, the specific wavelength ranges include 220-240 nm, 250-270 nm and 270-290 nm.

The pump 15 is provided in the accommodation space of the shell 10 to drive the aqueous solution to be measured to flow into the water inlet 111, through the sensing chamber 12, and out from the water outlet 112. More simply, the pump 15 allows the aqueous solution to be measured to flow inside the pipeline in the accommodation space.

The circuit board 16 is electrically connected with the light sensor 14 for receiving sensing signals sensed by the light sensor 14. The circuit board 16 is configured to calculate and determine the absorbance and the change in absorbance of a residual toxicant in the aqueous solution to be measured based on the sensing signal. In other words, through continuous detections via the light source emitter 13 and the light sensor 14, the sensing signals sensed can be used by the circuit board 16 to calculate the absorbance of a residual toxicant in the aqueous solution to be measured and its change in absorbance.

More particularly, when the aqueous solution to be measured is passing through the sensing chamber 12, a residual toxicant reacts with the light of a specific wavelength range, and the light sensor 14 senses the light of the specific wavelength range passing through the sensing chamber 12 to create a sensing signal.

The functionalities and operations of the circuit board 16 are described as follow. The circuit board 16 may include a drive unit, a reading unit, a memory unit, an arithmetic unit, a transmission unit, a display unit, etc. These units can be implemented in software or firmware. These units constitute a microcontroller (MCU) to achieve internal calculations, control, drive, transmission etc. inside the circuit board 16. Different circuit layouts can be provided based on different functionalities to achieve signal transmission with and control of the light source emitter 13, the light sensor 14, and the pump 15.

The drive unit is used for driving various components, including the turning on/off of the light source emitter 13. The drive unit controls the on/off of the light source emitter 13 since there may be a plurality of the light source emitters 13. Moreover, for continuous sensing, the light source emitter 13 may intermittently emit light, or the light sensor 14 may intermittently sense light. These can also be controlled by the drive unit. The drive unit may also drive other components if required.

The reading unit is used for reading a sensing signal at a fixed time interval. The reading unit primarily receives the sensing signals from the light sensor 14. As described before, the drive unit may allow the light source emitter 13 and the light sensor 14 to emit/sense light intermittently, so the reading unit may read the sensing signals at fixed time interval. This also has the effect of intermittent sampling.

The memory unit is used for storing the sensing signals. In order to enable subsequent data calculations, for example, to obtain an average, the sensing signals are thus stored in the memory unit.

The arithmetic unit is used for calculating a change in absorbance based on the sensing signals and calibration values to produce a detection result. The calculations of the change in absorbance of the aqueous solution to be measured and the calculations and determinations of degradation of monitored values obtained from continuous monitoring will be described in more details later.

The transmission unit is used for transmitting the detection result to an external device. The external device may be a corresponding apparatus or an electronic apparatus, such as a smartphone, a microcomputer, a tablet PC, a laptop etc. In an embodiment, the electronic apparatus may also send out the detection result via wireless transmission, and the corresponding apparatus may be, for example, a mount placed at the bottom of the residual toxicant detection device 1 (described in details later).

The display unit is used for displaying the detection result. For example, the display unit can be connected to a LED light or a display screen to display the detection result. In addition to the abovementioned components, the residual toxicant detection device 1 may further include other components to form a residual toxicant detection device 1 with functions such as water resistance, wireless charging, and the like.

In the case that the shell 10 includes the upper lid 101 and the lower lid 102, the residual toxicant detection device 1 may further include an O-ring 17. The O-ring 17 is positioned between the upper lid 101 and the lower lid 102, such that a tight seal is formed between the upper lid 101 and the lower lid 102 when they are joined together, preventing the aqueous solution to be measured from flowing into unexpected places in the residual toxicant detection device 1. Thus, the residual toxicant detection device 1 has waterproof functionality.

The residual toxicant detection device 1 may further include pipes 181, 182 and 183. These pipes 181, 182 and 183 connect the water inlet 111, the pump 15, the sensing chamber 12 and the water outlet 112, thereby forming a flow channel As shown in FIG. 1B, the water inlet 111 is connected to one end of the pipe 181, while the other end of the pipe 181 is connected to one end of the pump 15 via an adapter 241. The other end of the pump 15 is further connected with one end of the pipe 182 via an adapter 242, while the other end of the pipe 182 is connected to an inlet of the sensing chamber 12. An outlet of the sensing chamber 12 is connected to one end of the pipe 183 via an adapter 243. The other end of the pipe 183 is connected to the water outlet 112. As can be seen from the above, the flow channel of the water inlet 111, the pump 15, the sensing chamber 12 and the water outlet 112, as well as the pipes 181, 182 and 183 and the various adapters, form the path through which an aqueous solution to be measured will pass.

The residual toxicant detection device 1 may further include a battery 19 that provides the power required for the pump 15, the light source emitter 13, the light sensor 14 and the circuit board 16 to operate.

The residual toxicant detection device 1 may further include a first wireless charging module 20 positioned inside the accommodation space of the shell 10, which can be used in cooperation with the external device to charge the residual toxicant detection device 1 wirelessly. The power created by the first wireless charging module 20 can be stored in the battery 19.

The residual toxicant detection device 1 may further include a partitioning plate 21. The partitioning plate 21 is provided between the battery 19 and the first wireless charging module 20. The partitioning plate 21 is made of metal. In addition to separating the first wireless charging module 20 from the battery 19, the partitioning plate 21 also prevents the first wireless charging module 20 from interference during wireless charging. Moreover, another purpose of the partitioning plate 21 is allow the center of gravity of the residual toxicant detection device 1 to be at the bottom end, such that the residual toxicant detection device 1 can be held more or less upright in a half-floating and half-immersed or a completely immersed state in the aqueous solution to be measured when in use.

The residual toxicant detection device 1 may further include a first frame 221 and a second frame 222. The pump 15 and the sensing chamber 12 are sandwiched between the first frame 221 and the second frame 222. More specifically, when the first frame 221 and the second frame 222 are combined, the pump 15 and the sensing chamber 12 are secured inside to reduce abnormalities in measurements caused by movements of the pump 15 and the sensing chamber 12.

As shown in FIG. 1B, the circuit board 16 is provided at a side of the first frame 221, but the present disclosure is not limited as such. The circuit board 16 can also be provided next to the second frame 222. In addition, the light source emitter 13 can be provided on the first frame 221, while the light sensor 14 can be provided on the second frame 222.

In order to perform detections, the light source emitter 13 and the light sensor 14 are provided on two sides of the sensing chamber 12, that is, the light emitted by the light source emitter 13 will pass through the sensing chamber 12 to be sensed by the light sensor 14. However, the present disclosure is not limited as such. Alternatively, the light source emitter 13 and the light sensor 14 can be provided on the same side of the sensing chamber 12. In this case, the light emitted by the light source emitter 13 passes through the sensing chamber 12, and is then sensed by the light sensor 14 via a light reflector or other types of mirror.

In an embodiment, there is only one light source emitter 13 and only one light sensor 14, but the present disclosure is not limited as such. A plurality of light source emitters 13 and light sensors 14 can be provided around the sensing chamber 12.

Moreover, the residual toxicant detection device 1 may further include an LED indicator 23. It can be provided on the circuit board 16 for generating different colors based on the degree of absorbance. For example, red, yellow or green light can be shown based on the absorbance of the aqueous solution to be measured, such that a person performing the test is able to know the detection status of the aqueous solution to be measured.

When assembled, the O-ring 17 is fastened to the lower lid 102 via screws 27; the lower lid 102 and the upper lid 101 are secured together via screws 28; the first frame 221 and the second frame 222 are secured together via a screw 291; and the circuit board 16 is fastened to the first frame 221 via screws 292. The screws 27, 28, 291 and 292 can be used in conjunction with soft or hard gaskets to increase overall tightness.

In an embodiment, the residual toxicant detection device 1 may further include an outer case 25 and a bottom lid 26. The residual toxicant detection device 1, after the upper lid 101 and the lower lid 102 are combined, can be partially sheathed into the outer case 25. It provides aesthetic value while protecting the residual toxicant detection device 1. The bottom lid 26 envelops a portion of the outer case 25 and the lower lid 102 at the bottom of the residual toxicant detection device 1 to protect the bottom of the residual toxicant detection device 1 while preventing exposure of the screws 28.

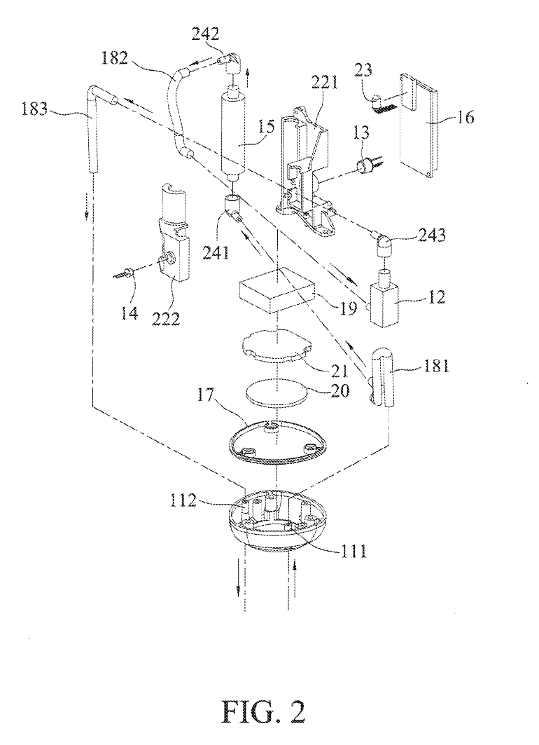

Referring to FIG. 2, a schematic diagram depicting the internal flow channel of the residual toxicant detection device in accordance with the present disclosure is shown. As shown, the components and combinations thereof are the same as those shown in FIG. 1B, in which the pump 15 and the sensing chamber 12 are positioned between the first frame 221 and the second frame 222, the light source emitter 13 is provided on the first frame 221, the light sensor 14 is provided on the second frame 222, and the circuit board 16 is positioned at a side of the first frame 221. In this embodiment, only the flow path of the aqueous solution to be measured inside the residual toxicant detection device 1 is illustrated.

The aqueous solution to be measured flows into the residual toxicant detection device 1 via the water inlet 111. The water inlet 111 is connected to the pipe 181, which is connected to the inlet of the pump 15 via the adapter 241. An outlet of the pump 15 is connected to the inlet of the pipe 182 via the adapter 242. The pipe 182, through a bend of, is connected to the inlet of the sensing chamber 12. As such, the aqueous solution to be measured flows into the sensing chamber 12 for optical sensing.

In an embodiment, the aqueous solution to be measured flows into the bottom of the sensing chamber 12, and flows out from the top of the sensing chamber 12. If water comes in from the top, air cavity may be formed in the sensing chamber 12. This may affect sensing. The aqueous solution to be measured, after coming out of the sensing chamber 12, flows into one end of the pipe 183 via the adapter 243 and then out of the water outlet 112 connected to the other end of the pipe 183. The above described is the flow path of the aqueous solution to be measured in the residual toxicant detection device 1 in accordance with the present disclosure.

As mentioned before, the change in absorbance of a residual toxicant in the aqueous solution to be measured is determined based on the sensing signals. The determining mechanism may include increasing an accumulated detection count when the change in absorbance of the aqueous solution to be measured is less than a threshold, and sending a detection result when the accumulated detection count is equal to or greater than a predetermined value. In an embodiment, a calibration value (or baseline value) can be obtained before the detection, which is the detection signal value obtained when there is no test subject. Based on the calibration value and a detection value (a signal value obtained by sensing of the light sensor) of a toxicant in the aqueous solution obtained from the sensing signals, the change in absorbance of the residual toxicant in the aqueous solution to be measured can be calculated.

Two determining methods are described below. In the first method, during continuous monitoring, if the change in absorbance of the aqueous solution to be measured is less than a predetermined threshold, the value of the accumulated detection count is increased by 1. At this time, the change in absorbance is small, indicating that the residual toxicant in the aqueous solution to be measured has been reduced to a certain extent. However, in order to avoid misjudgment of a single detection, the accumulated detection count is used to accumulate a number of these occurrences. In other words, each time the change in absorbance is less than the threshold, the accumulated detection count is incremented by 1; and when the accumulated detection count has reached a predetermined value, the removal of the residual toxicants in the aqueous solution to be measured has more or less stopped.

The second determining method involves that generation of a detection result once the change in absorbance of the aqueous solution to be measured is less than a threshold continuously for a predetermined number of times. In an embodiment, the change in absorbance being less than the threshold may occur continuously or non-continuously. In previous embodiment, the end of the removal of residual toxicants in the aqueous solution to be measured is determined when the change in absorbance being less than the threshold has occurred a predetermined number of times (can be continuous or non-continuous). In this embodiment, the end of the removal of residual toxicants in the aqueous solution to be measured is determined only when the change in absorbance being less than the threshold has continuously occurred for a predetermined number of times, for example, five times.

With the detection mechanism above, through continuous detection, the amount of residual toxicants in an aqueous solution can be determined by determining the absorbance of the aqueous solution, and the end of the removal of the residual toxicants can be determined when the change in absorbance satisfies or continuously satisfies an expected condition. Therefore, the detection method of the present disclosure is simple to use without the need for comparison of multiple toxicants in a large database, facilitating use by the general public.

The calculations of the change in absorbance of the aqueous solution to be measured and the calculations and determining of degradation of monitored values obtained from continuous monitoring are described in more details below.

As described before, a calibration value (or called baseline value measured in voltage, power etc.) can be obtained before detection, which is the detected signal value measured when there is no test subject. The absorbance can then be obtained by comparing the calibration value and the detected value. The greater the difference between the detected value and the calibration value is, the larger the absorbance becomes. The closer the detected value is to the calibration value, the smaller the absorbance becomes. An example of how the absorbance is calculated is given by the equation (1) below.

Absorbance=−20×log(Detected Value/Calibration Value) eqn. (1),

wherein the negative sign of −20 is necessary, and 20 is an adjustable value. The detected value is the signal value obtained by sensing of the light sensor (measured in terms of voltage, power etc.). From the equation (1) above, it is clear that the larger the absorbance, the greater the difference between the detected value and the calibration value, and the smaller the absorbance, the smaller the difference between the detected value and the calibration value. The equation (1) is only exemplary, and the calculations of the absorbance are not limited to this equation.

In addition, the calculations involve degradation calculations and determining. The present disclosure may perform detections at fixed time interval. For example, after X absorbance calculation results are obtained. More particularly, when there are X data, an average is first obtained from the X data, then the average value is subtracted from each of the X data to obtain variations of the X data with respect to the average. For example, an average can be taken for every 10 data.

When the variation is less than a threshold Y, indicating that the variation is not large compared to the average and is within an expected range, the accumulated detection count is incremented by 1. For example, the value of Y can be set according to the needs, for example, Y can be 0.05 dB. Finally, when the accumulated detection count is equal to Z, the removal of residual toxicants in the test subject is determined to have ended. For example, when the accumulated detection count has reached 5, then the removal of residual toxicants in the test subject is determined to have ended. In an embodiment, this accumulated detection count is continuous accumulation. When the continuously accumulated detection count has reached Z, then the removal of residual toxicants in the test subject is determined to have ended.

The present disclosure does not need to know in advance the type of specimen (toxicant) it is. By detecting the change in absorbance produced in the aqueous solution to be measured, the degree of removal of the residual toxicants on the test subject is known. As the absorbance of the aqueous solution to be measured keeps changing during washing of the test subject, the present disclosure can perform continuous detections. When the change in absorbance is less than a threshold for a predetermined number of times or continuously for a predetermined number of times, then the residual toxicants of the test subject are thought to be less than the predefined standard.

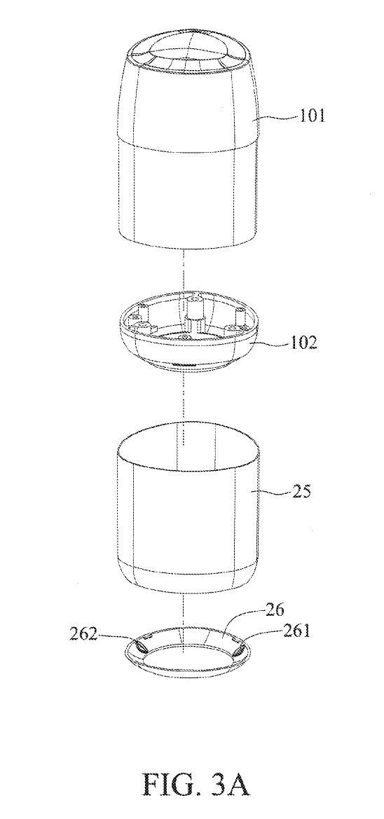

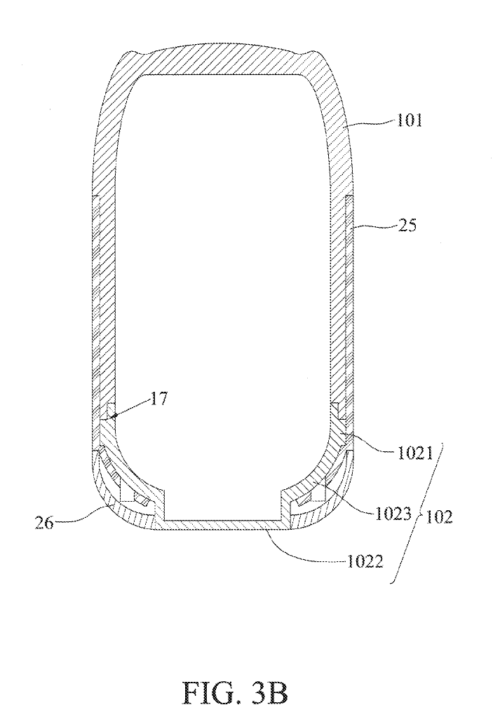

Referring to FIGS. 3A and 3B, schematic diagrams depicting the outer casing of the residual toxicant detection device in accordance with present disclosure are shown. As shown in FIG. 3A, the upper lid 101 and the lower lid 102 are combined and sheathed by the outer case 25. Thereafter, the bottom lid 26 is installed at the bottom of the residual toxicant detection device to cover portions of the outer case 25 and portions of the lower lid 102. There are two holes 261 and 262 provided on the bottom lid 26 to match the water inlet 111 and the water outlet 112 of the lower lid 102, respectively.

The thickness of the outer case 25 can be 0.5 mm. In an embodiment, the outer case 25 can be made of metal, but the present disclosure is not limited to this. The upper lid 101 and lower lid 102 made of plastic can be joined with the outer case 25 by using an epoxy glue (e.g., AB glue).

As shown in FIG. 3B, when the upper lid 101 is combined with the lower lid 102, the O-ring 17 is provided between the upper lid 101 and the lower lid 102 to increase tightness and prevent the aqueous solution to be measured from flowing in. Once the upper lid 101 is combined with the lower lid 102, the outer case 25 covers a portion of the upper lid 101 and a portion of the lower lid 102, wherein the outer case 25 does not cover the bottom of the lower lid 102, where there is a wireless charging port. The bottom lid 26 covers a portion of the outer case 25 and a portion of the lower lid 102 after the outer case 25 is installed.

The lower lid 102 includes a side face 1021, a bottom face 1022 and a bevel 1023 adjoining the side face 1021 and the bottom face 1022. In an embodiment, the water inlet and outlet are provided on the bevel 1023, but the present disclosure is not limited as such. The water inlet and outlet can also be provided on the side face 1021. If the fact that the residual toxicant detection device in use may be upright due to center of gravity is taken into account and the lower lid 102 of the residual toxicant detection device may touch the bottom, the water inlet and outlet are preferably not provided on the bottom face 1022.

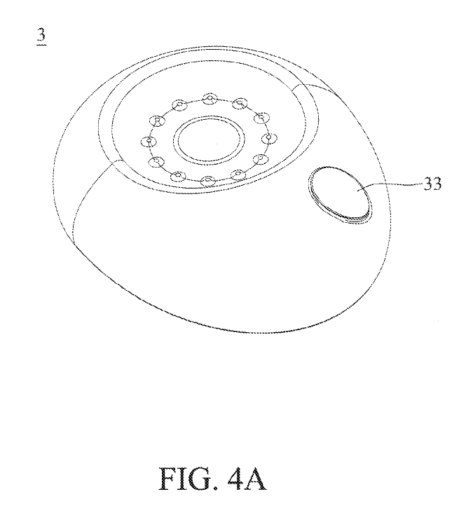

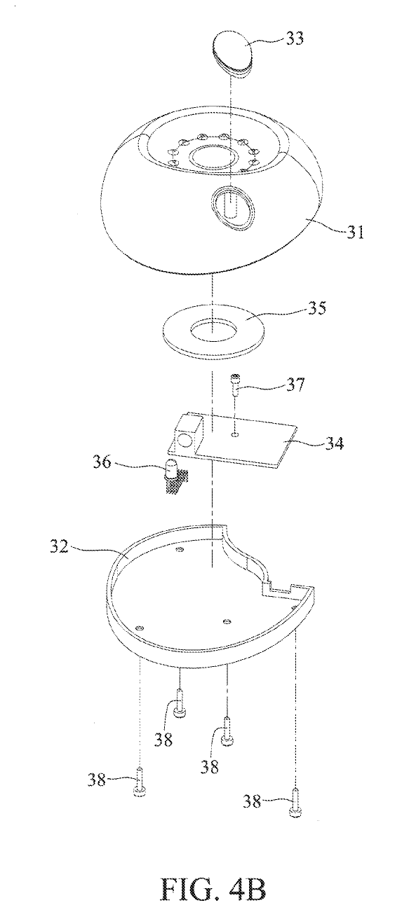

Referring to FIGS. 4A and 4B, schematic diagrams depicting the overall appearance and an exploded view of a mount for the residual toxicant detection device in accordance with the present disclosure are shown. Considering the need for holding and wireless charging of the residual toxicant detection device when not in use, an external mount can be provided for holding and wireless charging of the residual toxicant detection device 1.

As shown in FIG. 4A, an appearance of the mount 3 is shown. A recessed portion is provided in the middle of the mount 3 for receiving the residual toxicant detection device 1 (outer case) and carrying out wirelessly charging. The mount 3 includes a button 33 to allow a user to activate the residual toxicant detection device 1.

As shown in FIG. 4B, an exploded view of the mount 3 is shown. The mount 3 includes a mount upper lid 31, a mount lower lid 32, the button 33, a mount circuit board 34 and a second wireless charging unit 35.

The mount upper lid 31 is fastened to the mount lower lid 32 via screws 38. The button 33 is provided on the mount upper lid 31. The mount circuit board 34 is fastened to the mount lower lid 32 via a screw 37. The mount circuit board 34 and the second wireless charging unit 35 are received in the space formed after the mount upper lid 31 and the mount lower lid 32 are combined.

The second wireless charging unit 35 can be provided above the mount circuit board 34 for use in cooperation with the first wireless charging module 20 to carry out wireless charging. The distance between the first wireless charging module of the residual toxicant detection device 20 of the residual toxicant detection device and the second wireless charging unit 35 of the mount 3 is about 5 mm. Wireless charging techniques are well known to those skilled in the art, and will not be repeated.

The mount 3 further includes a second LED indicator 36. The second LED indicator 36 shows different colors based on the degree of absorbance. Simply put, the residual toxicant detection device senses and obtains absorbance associated with the residual toxicants in the aqueous solution to be measured. In addition to the LED indicator 23 of the residual toxicant detection device providing indication, the residual toxicant detection device also transmits signals to the mount circuit board 34, and the second LED indicator 36 connected to the mount circuit board 34 also indicates the degree of absorbance. During detection, if the user cannot see the LED indicator 23, he/she can look at the second LED indicator 36 on the mount 3. The second LED indicator 23 and the second LED indicator 36 should show the same color.

It is know from the above that the mount 3 includes functions such as charging, controlling the activation of the residual toxicant detection device and receiving signals from the residual toxicant detection device, wherein the battery of the residual toxicant detection device is charged wirelessly, and wireless communication with the residual toxicant detection device is made via Bluetooth or Wi-Fi, for example.

In addition, the mount 3 may be equipped just wireless charging ability, but not the ability to control the residual toxicant detection device. The control of the operations of the residual toxicant detection device can be achieved by using an application installed in a mobile phone, for example.

The hand-held residual toxicant detection device according to the present disclosure is capable of detecting change in absorbance of residual toxicants in an aqueous solution to be measured through an optical sensing mechanism. When in use, the residual toxicant detection device is placed in the aqueous solution to be measured, which then absorbs the aqueous solution to be measured for measurement and shows the detection result through LED indicators. The residual toxicant detection device is designed to be a single integral device with waterproof and wireless charging abilities, and is suitable for general home testing.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A device for detecting a residual toxicant in an aqueous solution to be measured, the device comprising:
a shell including an accommodation space;
a water inlet formed on the shell and in communication with the accommodation space, and configured for the aqueous solution to be measured to flow into the accommodation space;
a sensing chamber provided in the accommodation space and configured for the aqueous solution to be measured to flow therein;
a light source emitter provided near the sensing chamber and configured to emit light passing through the sensing chamber;
a light sensor provided near the sensing chamber and configured to sense the light passing through the sensing chamber to produce a sensing signal;
a water outlet formed on the shell and in communication with the accommodation space, and configured for the aqueous solution to be measured to flow out of the accommodation space; and
a circuit board electrically connected with the light sensor and configured to receive the sensing signal.

2. The device of claim 1, wherein the circuit board is configured to calculate absorbance of the residual toxicant in the aqueous solution to be measured based on the sensing signal.

3. The device of claim 1, wherein the shell includes an upper lid and a lower lid.

4. The device of claim 3, further comprising an O-ring provided between the upper lid and the lower lid.

5. The device of claim 3, wherein the lower lid includes a side face, a bottom face and a bevel adjoining the side face and the bottom face, and the water inlet and the water outlet are provided on the bevel.

6. The device of claim 1, further comprising a pump provided in the accommodation space and configured to drive the aqueous solution to be measured to flow.

7. The device of claim 6, wherein the circuit board is configured to calculate a change in absorbance of the residual toxicant in the aqueous solution to be measured by continuously detecting the flowing aqueous solution to be measured via the light source emitter and the light sensor.

8. The device of claim 6, further comprising a plurality of pipes configured to connect the water inlet, the pump, the sensing chamber and the water outlet to form a flow channel.

9. The device of claim 6, further comprising a battery configured to provide power required for the pump, the light source emitter, the light sensor and the circuit board.

10. The device of claim 9, further comprising a first wireless charging module provided in the accommodation space and configured to generate and store the power in the battery.

11. The device of claim 10, further comprising a partitioning plate provided between the battery and the first wireless charging module.

12. The device of claim 6, further comprising a first frame and a second frame, with the pump and the sensing chamber provided between the first frame and the second frame.

13. The device of claim 12, wherein the circuit board is provided at a side of the first frame or the second frame.

14. The device of claim 1, wherein at least one of the light source emitter and the light sensor are plural in number.

15. The device of claim 1, wherein the light source emitter and the light sensor are provided on opposite sides of the sensing chamber.

16. The device of claim 1, further comprising an LED indicator provided on the circuit board and configured to show different colors based on absorbance of the residual toxicant.

17. The device of claim 1, further comprising a mount configured to hold the shell.

18. The device of claim 17, wherein the mount includes a second wireless charging module, and a first wireless charging module in the device and the second wireless charging module are configured to charge the device wirelessly.

19. The device of claim 17, wherein the mount includes a button configured to activate the device.
